(19) United States
(12) Patent Application Publication
(10) Pub. No.: US 2021/0259604 A1
(43) Pub. Date: Aug. 26, 2021

(54) Method, Device, Computer Program and Computer Program Product for Detecting the Attentiveness of the Driver of a Vehicle

(71) Applicant: BAYERISCHE MOTOREN WERKE AKTIENGESELLSCHAFT, Muenchen (DE)

(72) Inventors: Florian BADE, Muenchen (DE); Martin BUCHNER, Muenchen (DE); Maurice KOLFF, Sleeuwijk (NL); Joris WOLTERS, Amersfoort (NL)

(73) Assignee: Bayerische Motoren Werke Aktiengesellschaft, Muenchen (DE)

(21) Appl. No.: 17/053,932

(22) PCT Filed: May 27, 2019
(86) PCT No.: PCT/EP2019/063596

§ 371 Date: Nov. 9, 2020

(30) Foreign Application Priority Data
Sep. 3, 2018 (DE) 10 2018 214 935.6

(51) Int. Cl. A61B 5/18; A61B 5/16

(52) U.S. Cl. CPC A61B 5/18; G06K 9/00845; A61B 2503/22; A61B 5/163

(57) ABSTRACT

In a method for determining the attentiveness of the driver of a vehicle, the inclination of the driver's head is detected. On the basis of the inclination of the head it is determined whether the head is directed downwards. If it has been detected that the head is directed downwards, the size of the opening of at least one of the driver's eyes is continuously detected. Depending on the size of the eyelid opening it is determined whether the driver has directed a reference glance at a carriageway. Depending on the determination as to whether the driver has directed a reference glance at the carriageway it is determined whether the driver is attentive.

### BACKGROUND AND SUMMARY OF THE INVENTION

The invention relates to a method for determining an attentiveness of a driver of a vehicle. The invention relates moreover to a device for determining an attentiveness of a driver of a vehicle. The invention relates moreover to a computer program and a computer-program product.

In modern vehicles, camera-based systems are used for the purpose of determining attentiveness. These systems require, for instance, very high-resolution and expensive cameras, in order to determine a direction of view, depending on a pupil.

The object underlying the invention is to enable a simple and reliable determination of an attentiveness of a driver of a vehicle.

The object is achieved by the features of the independent claims. Advantageous configurations are characterized in the dependent claims.

According to a first aspect, the invention is distinguished by a method for determining an attentiveness of a driver of a vehicle. The invention is distinguished moreover by a device for determining an attentiveness of a driver of a vehicle, the device being designed to execute the method for determining an attentiveness of a driver of a vehicle.

In the case of the method for determining an attentiveness of a driver of a vehicle, an inclination of the head of the driver is detected. Depending on the inclination of the head, it is determined whether the head is directed downward. If it was determined that the head is directed downward, a magnitude of an eyelid opening of at least one eye of the driver is continually detected. Depending on the magnitude of the eyelid opening, it is determined whether the driver has taken a reference look at a roadway. Depending on the determination of whether the driver has taken a reference look at a roadway, it is determined whether the driver is attentive.

The method is based on the determination of the inclination of the head and on the determination of the magnitude of an eyelid opening. These two variables can be detected with a simple camera system and do not require a very high-resolution and expensive camera.

If, however, the attentiveness were to be determined only in a manner depending on the inclination of the head, this could lead to the following error. If a driver is deliberately looking down—in order, for instance, to operate a navigation device—but is taking a checking look—that is to say, a reference look—at the roadway at regular intervals, this driver is actually attentive. In the case of a system that only takes the inclination of the head into consideration, such a driver would, however, be rated as inattentive, and an appropriate measure would be taken.

By virtue of the above method, consequently fewer misclassifications are carried out, without requiring a very high-resolution and expensive camera. Consequently, the method enables a simple and reliable determination of the attentiveness of the driver.

The determination of whether the head is directed downward can be undertaken, for instance, by comparing with a threshold value. If the inclination of the head is greater than the threshold value, the head is inclined downward; if the inclination of the head is less, the head is inclined upward.

According to an optional configuration, a timer is started after the determination that the head is directed downward. The driver is classified as attentive if the reference look is taken prior to expiration of the timer.

The timer is, for instance, within the range between 2 seconds and 4 seconds, for instance 3 seconds.

If the timer expires, the driver is classified as inattentive, and an appropriate measure can be taken. Such measures include, for instance, a display of a warning message, an acoustic warning message, a haptic warning message, for instance by a vibration of the steering wheel.

If the reference look is taken prior to expiration of the timer, the driver is classified as attentive. After this, the timer can be restarted, and the magnitude of the eyelid opening can continue to be monitored, in order to detect further reference looks, for instance until the head has been directed upward again.

According to a further optional configuration, a moving average of the magnitude of the eyelid opening is determined. A percentage eyelid opening is determined, by the continually detected magnitude of the eyelid opening being divided by the moving average of the magnitude of the eyelid opening. Depending on the percentage eyelid opening, it is determined whether the driver has taken a reference look at a roadway.

By the percentage eyelid opening being determined, a normalized magnitude of the eyelid opening is created. By this means, the method can be carried out for various people, without a calibration having to be carried out in each instance.

The moving average of the magnitude of the eyelid opening has, for instance, a filter width of several minutes, for instance 2 minutes to 3 minutes.

According to a further optional configuration, the determination of whether the driver has taken a reference look at a roadway is only carried out if an initial magnitude of the percentage eyelid opening lies below a predetermined threshold value.

By this means, a contribution is made to ensuring that the method is only carried out if the driver is actually looking down and not just inclining his/her head without looking down.

According to a further optional configuration, the determination of whether the driver has taken a reference look at a roadway is only carried out if the moving average of the magnitude of the eyelid opening is greater than a predetermined threshold value.

By this means, a contribution is made to ensuring that at the start of a detection the moving average firstly has to build up and cannot be taken for a reliable detection in this phase. Furthermore, in addition a check can also be made as to whether the vehicle is moving, so that detections at a standstill are not taken into consideration for the purpose of determining the moving average, since these are irrelevant for a recognition of attentiveness.

According to a further optional configuration, if two eyes are detected, a magnitude of an eyelid opening is continually detected in each instance for both eyes. A mean value is continually determined from both variables. Depending on the mean value, it is determined whether the driver has taken a reference look at a roadway.

In principle, the method works with only one eye, but if both eyes are detected the eyelid openings of both eyes can also be used, in order to enable a more reliable determination of the recognition of attentiveness.

According to a second aspect, the invention is distinguished by a method for determining an attentiveness of a driver of a vehicle. The invention is distinguished moreover by a device for determining an attentiveness of a driver of a vehicle, the device being designed to execute the method for determining an attentiveness of a driver of a vehicle.

In the case of the method for determining an attentiveness of a driver of a vehicle, an inclination of the head of the driver is detected. Depending on the inclination of the head, it is determined whether the head is directed downward. If it was determined that the head is not directed downward, a magnitude of an eyelid opening of at least one eye of the driver is continually detected. Depending on the magnitude of the eyelid opening, it is determined whether the driver is looking down. If it was determined that the driver is looking down, depending on the magnitude of the eyelid opening it is determined whether the driver has taken a reference look at a roadway. Depending on the determination of whether the driver has taken a reference look at a roadway, it is determined whether the driver is attentive.

The method of the second aspect is likewise based on the detection of the inclination of the head and on the detection of the magnitude of an eyelid opening. These two variables can be determined with a simple camera system and do not require a very high-resolution and expensive camera.

If, however, the attentiveness were to be determined only in a manner depending on the inclination of the head, this could lead to the following error. If a driver is not inclining his/her head downward but is looking down with his/her eyes—in order, for instance, to operate a navigation device—and is not taking a checking look—that is to say, a reference look—at the roadway, this driver is actually inattentive. In the case of a system that takes only the inclination of the head into consideration, such a driver would, however, be rated as attentive, and no appropriate measure would be taken.

The method of the second aspect enables this error to be reduced. By virtue of the above method, consequently fewer misclassifications are carried out, without requiring a very high-resolution and expensive camera. Consequently, the method enables a simple and reliable determination of the attentiveness of the driver.

In principle, the determination of the reference look is undertaken as in the method of the first aspect. Consequently, the same advantages apply to the method of the second aspect as to the method of the first aspect, and the same optional configurations are applicable as to the method of the first aspect.

According to a further aspect, the invention is distinguished by a computer program, the computer program being designed to carry out the method of the first aspect or of the second aspect.

According to a further aspect, the invention is distinguished by a computer-program product which includes executable program code, the program code executing the method of the first aspect or of the second aspect upon execution by a data-processing device.

The computer-program product includes, in particular, a medium capable of being read by the data-processing device, on which the program code has been stored.

Embodiments of the invention are elucidated in more detail in the following with reference to the schematic drawings.

### BRIEF DESCRIPTION OF THE DRAWINGS

FIG. 1 is a flow chart for determining an attentiveness of a driver of a vehicle and

FIG. 2 is a further flow chart for determining an attentiveness of a driver of a vehicle.

### DETAILED DESCRIPTION OF THE DRAWINGS

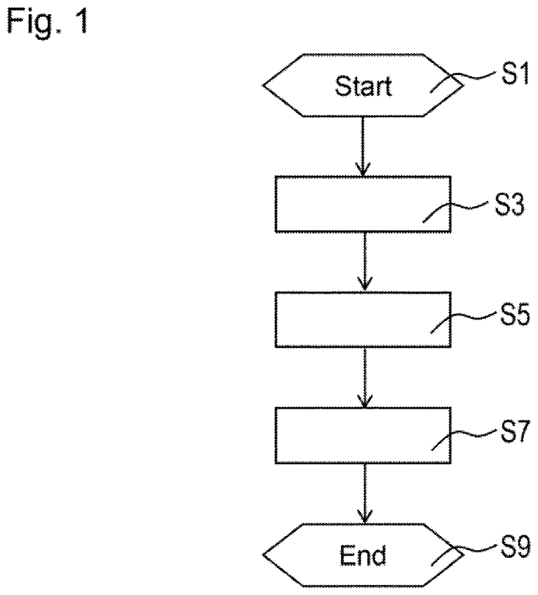

FIG. 1 shows a flow chart of a program for determining an attentiveness of a driver of a vehicle.

The program can be run by a device. The device is arranged in a vehicle, for instance.

For this purpose the vehicle exhibits a camera which may also be part of the device.

The device may also be designated as a device for determining an attentiveness of a driver of a vehicle.

For this purpose the device exhibits, in particular, an arithmetic unit, a program-and-data memory, and also, for instance, one or more communication interfaces. The program-and-data memory and/or the arithmetic unit and/or the communication interfaces may have been arranged in one structural unit and/or distributed to several structural units.

For this purpose, in particular a program for determining an attentiveness of a driver of a vehicle has been stored in the program-and-data memory of the device.

The program is started in a step S1, in which variables can be initialized where appropriate.

An inclination of the head of the driver is detected in a step S3.

If it was detected that the head is directed downward, the program is continued in a step S5.

In step S5 a magnitude of an eyelid opening of at least one eye of the driver is continually detected.

In a step S7, depending on the magnitude of the eyelid opening, it is determined whether the driver has taken a reference look at a roadway and, depending on the determination of whether the driver has taken a reference look at a roadway, it is determined whether the driver is attentive.

The program is subsequently terminated in a step S9 and can, where appropriate, be restarted in step S1.

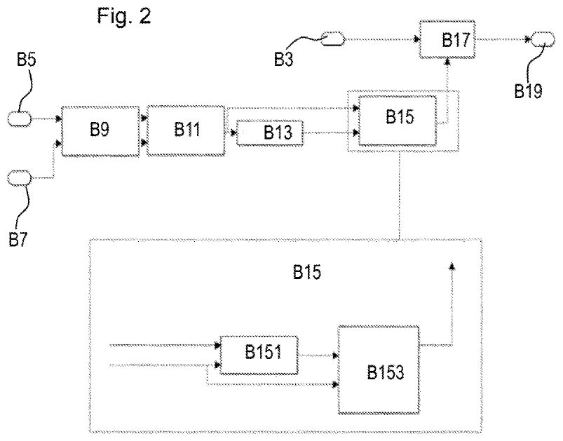

Steps S5 and S7 can be divided up into the following individual blocks (see FIG. 2).

After the determination that the head is directed downward, a timer (block B3) is started.

By way of further input variables, a magnitude of the eyelid opening of a left eye (block B5) and a magnitude of the eyelid opening of a right eye (block B7) are made available.

These magnitudes of the eyelid opening are optionally corrected, by peaks (which, for instance, result from measurement errors) being filtered out (block B9).

If two eyes are detected (that is to say, if, for instance, both magnitudes of the eyelid opening are >0), a mean value, for instance, is determined from both variables and is used in the further calculation as “magnitude of the eyelid opening”; if only one eye is detected (that is to say, if, for instance, one magnitude of the eyelid opening=0 and the other is >0), the magnitude of the eyelid opening of this eye is used in the further calculation as “magnitude of the eyelid opening” (block B11).

A moving average of the magnitude of the eyelid opening is determined (block B13). The moving average of the magnitude of the eyelid opening and the magnitude of the eyelid opening are supplied to a block B15 as input variables.

A percentage eyelid opening is determined, by the continually determined magnitude of the eyelid opening being divided by the moving average of the magnitude of the eyelid opening (block B151).

The percentage eyelid opening and the moving average of the magnitude of the eyelid opening are supplied to a block B153 as input variables.

In block B153 it is determined whether a reset of the timer is to be carried out.

First of all, a reference look is determined if a difference in the percentage eyelid opening within a predetermined time-interval is greater than a predetermined threshold value of the increase. The predetermined time-interval is, for instance, the interval between two successive measured values. Accordingly, simply the difference between two successive values of the percentage eyelid opening is derived, and this difference is compared with a predetermined threshold value. If the difference is greater than the threshold value, a reset of the timer can be undertaken.

Optionally, however, in order to enhance the robustness of the method several further conditions for a reset of the timer can be logically linked with “and”:

In particular, in order to exclude blinking, the difference between two successive values of the percentage eyelid opening can be compared with a predetermined upper threshold value, and only if the difference is less than the threshold value can a logical “1” be output.

Furthermore, a check can be made as to whether an initial magnitude of the percentage eyelid opening lies below a predetermined threshold value. For this purpose, for instance, a value of the percentage eyelid opening after the head was inclined downward, or a value at a predetermined point in time, for instance 0.2 seconds to 0.8 seconds ago, is compared with the threshold value which, for instance, lies within a range between 0% and 30%, for instance is 30%. If the value is greater than the threshold value, a logical “1” is output. By this means, a contribution is made to ensuring that the method is only carried out if the driver is also really looking down and is not just inclining his/her head without looking down.

Furthermore, a check can be made as to whether the moving average of the magnitude of the eyelid opening is greater than a predetermined threshold value. If the value is greater than the threshold value, a logical “1” is output. By this means, a contribution is made to ensuring that at the start of a detection the moving average firstly has to build up and cannot be taken for a reliable detection in this phase. Furthermore, in addition a check can also be made as to whether the vehicle is moving, so that detections at a standstill are not taken into consideration for the purpose of determining the moving average, since these are irrelevant for a recognition of attentiveness.

If a logical “1” is now output from block 153, a reset of the timer is executed in block B17.

The timer is output in a block B19.

If the timer expires, the driver is classified as inattentive, and an appropriate measure can be taken. Such measures include, for instance, a display of a warning message, an acoustic warning message, a haptic warning message, for instance by a vibration of the steering wheel. The expiration may occur in two ways: If the timer is an incrementing timer, the exceeding of a threshold value may be understood as expiration. If the timer is a countdown timer, the reaching of zero may be understood as expiration. The timer is, for instance, within the range between 2 seconds and 4 seconds, for instance 3 seconds.

All the predetermined variables and/or threshold values described above can be determined, for instance, in preliminary tests in respect of test persons.

By virtue of the above method it can be ensured that fewer misclassifications are carried out, without requiring a very high-resolution and expensive camera. Consequently, the method enables a simple and reliable determination of the attentiveness of the driver.

The above method can, in slightly modified form, also be carried out as follows.

An inclination of the head of the driver is detected. Depending on the inclination of the head, it is determined whether the head is directed downward. If it was determined that the head is not directed downward, a magnitude of an eyelid opening of at least one eye of the driver is continually detected. Depending on the magnitude of the eyelid opening, it is determined whether the driver is looking down. If it was determined that the driver is looking down, depending on the magnitude of the eyelid opening it is determined whether the driver has taken a reference look at a roadway. Depending on the determination of whether the driver has taken a reference look at a roadway, it is determined whether the driver is attentive.

In principle, the determination of a reference look is undertaken as in steps S3 and S5. Consequently, the same description of steps S3 and S5, or of the blocks of FIG. 2, applies to the method.

1.-11. (canceled)

12. A method for determining an attentiveness of a driver of a vehicle, in which an inclination of the head of the driver is detected, the method comprising:
(i) depending on the inclination of the head, determining whether the head is directed downward;
(ii) when a determination is made that the head is directed downward,
(a) continually detecting a magnitude of an eyelid opening of at least one eye of the driver is continually detected,
(b) depending on the magnitude of the eyelid opening, determining whether the driver has taken a reference look at a roadway, and
(c) depending on the determination of whether the driver has taken a reference look at a roadway, determining whether the driver is attentive.

13. The method according to claim 12, further comprising:
starting a timer after the determination that the head is directed downward, and
classifying the driver as attentive if the reference look is taken prior to expiration of the timer.

14. The method according to claim 12, wherein
a moving average of the magnitude of the eyelid opening is determined,
a percentage eyelid opening is determined by the continually determined magnitude of the eyelid opening being divided by the moving average of the magnitude of the eyelid opening, and
depending on the percentage eyelid opening, determining whether the driver has taken a reference look at a roadway.

15. The method according to claim 14, wherein
a determination is made that the driver has taken a reference look at a roadway if a difference in the percentage eyelid opening within a predetermined time-interval is greater than a predetermined threshold value of the increase.

16. The method according to claim 14, wherein
the determination of whether the driver has taken a reference look at a roadway is only carried out if an initial magnitude of the percentage eyelid opening lies below a predetermined threshold value.

17. The method according to claim 15, wherein
the determination of whether the driver has taken a reference look at a roadway is only carried out if the moving average of the magnitude of the eyelid opening is greater than a predetermined threshold value.

18. The method according to claim 12, wherein, when two eyes are detected:
a magnitude of an eyelid opening is continually detected for both eyes,
a mean value is continually determined from both variables,
depending on the mean value, it is determined whether the driver has taken a reference look at a roadway.

19. A method for determining an attentiveness of a driver of a vehicle, comprising:
detecting an inclination of the head of the driver;
depending on the inclination of the head, determining whether the head is directed downward;
when a determination is made that the head is not directed downward,
(a) continually detecting a magnitude of an eyelid opening of at least one eye of the driver,
(b) depending on the magnitude of the eyelid opening, determining whether the driver is looking downward,
(c) when a determination is made that the driver is looking downward, depending on the magnitude of the eyelid opening determining whether the driver has taken a reference look at a roadway, and
depending on the determination of whether the driver has taken a reference look at a roadway, determining whether the driver is attentive.

20. A device for determining an attentiveness of a driver of a vehicle, comprising:
a processor configured to execute the acts of:
detecting an inclination of the head of the driver;
depending on the inclination of the head, determining whether the head is directed downward;
when a determination is made that the head is directed downward,
(a) continually detecting a magnitude of an eyelid opening of at least one eye of the driver is continually detected,
(b) depending on the magnitude of the eyelid opening, determining whether the driver has taken a reference look at a roadway, and
(c) depending on the determination of whether the driver has taken a reference look at a roadway, determining whether the driver is attentive.

21. A computer product comprising a non-transitory computer readable medium having stored thereon program code that, when executed by one or more processors, carries out the acts of:
detecting an inclination of the head of the driver;
depending on the inclination of the head, determining whether the head is directed downward;
when a determination is made that the head is directed downward,
(a) continually detecting a magnitude of an eyelid opening of at least one eye of the driver is continually detected,
(b) depending on the magnitude of the eyelid opening, determining whether the driver has taken a reference look at a roadway, and
(c) depending on the determination of whether the driver has taken a reference look at a roadway, determining whether the driver is attentive.
